(19) United States
(12) Patent Application Publication
(10) Pub. No.: US 2026/0249013 A1
(43) Pub. Date: Aug. 27, 2026

(54) MEDICAMENT DELIVERY DEVICE

(71) Applicant: Genzyme Corporation, Cambridge, MA (US)

(72) Inventors: Alexander Hee-Hanson, Melbourn (GB); Thomas Lever, Melbourn (GB); Michael Parrott, Melbourn (GB); Robert Wilson, Melbourn (GB); Haiming Wu, Cambridge, MA (US)

(21) Appl. No.: 19/064,399

(22) Filed: Feb. 26, 2025

(51) Int. Cl. A61M 5/20; A61M 5/32

(52) U.S. Cl. CPC A61M 5/2033; A61M 5/3202; A61M 5/3243; A61M 2005/2073

(57) ABSTRACT

A medicament delivery device for injecting medicament is described. The medicament delivery device has a body having a proximal end and a distal end, a needle for injecting medicament, an actuation member which is movable relative to the body from a first position to a second position for dispensing medicament from the needle and a cap removably attached to the body. When the cap is attached to the body, the cap covers the distal end of the body for preventing access to the needle. The cap is configured to prevent movement of the actuation member from the first position to the second position when the cap is attached to the body.

### TECHNICAL FIELD

The present disclosure relates to a medicament delivery device, a method of preparing a medicament delivery device for use and a method of using a medicament delivery device.

### BACKGROUND

Medicament delivery devices, such as auto-injectors, are known in the art for dispensing medicament to an injection site of a patient. In some cases, the needle actuator can be depressed accidentally, for example, before the device is ready and/or in position for use. Depressing the needle actuator accidentally can cause a dose of medicament to be unintentionally dispensed. This can lead to a waste of medicament.

### SUMMARY

According to a first aspect of the present disclosure, there is provided a medicament delivery device for injecting medicament, wherein the medicament delivery device comprises a body having a proximal end and a distal end; a needle for injecting medicament; an actuation member which is movable relative to the body from a first position to a second position for dispensing medicament from the needle; and a cap which is removably attached to the medicament delivery device, wherein the cap is configured to prevent movement of the actuation member from the first position to the second position when the cap is attached to the medicament delivery device.

The cap may comprise a proximally-extending portion which is configured to prevent movement of the actuation member from the first position to the second position.

The medicament delivery device may further comprise an axially-extending component which is configured to engage the proximally-extending portion to prevent movement of the actuation member from the first position to the second position.

The actuation member may comprise the axially-extending component.

The axially-extending component and the actuation member may be separate components.

The medicament delivery device may further comprise an axially-extending component which is movable relative to the body and a radially-extending surface, wherein the radially-extending surface is configured to engage the axially-extending component for preventing distal movement of the axially-extending component.

The cap may comprise a proximally-extending portion which is configured to engage the axially-extending component on the opposite side of the axially-extending component to the radially-extending surface.

The axially-extending component may be a first axially-extending component, and wherein the device may further comprise a second axially-extending component which is configured to engage the first axially-extending component on the opposite side of the first axially-extending component to the radially-extending surface, wherein the second axially-extending component is movable relative to the body.

The cap may comprise a proximally-extending portion which is configured to engage the second axially-extending component when the cap is attached to the medicament delivery device.

One of the proximally-extending portion and the second axially-extending component may comprise a protrusion and the other of the proximally-extending portion and the second axially-extending component may comprise a recess and wherein the protrusion is received in the recess when the cap is attached to the medicament delivery device.

The cap may be configured to engage a distal-facing surface of the second axially-extending component to prevent the second axially-extending component from moving distally when the cap is attached to the medicament delivery device.

The medicament delivery device may further comprise a bias configured to bias the second axially-extending component in the distal direction.

The axially-extending component may comprise a protrusion which extends radially from the axially-extending component, and wherein the radially-extending surface is configured to engage the protrusion.

The axially-extending component may be flexible and wherein when the actuation member moves from the first position to the second position then the axially-extending component moves distally and is deflected radially by the engagement of the axially-extending component and the radially-extending surface.

The proximally-extending portion may be configured to abut a distal-facing surface of the axially-extending component to prevent the actuation member from moving from the first position to the second position.

The actuation member may comprise a button configured to be pressed to move the actuation member from the first position to the second position.

The device may comprise a dispensing mechanism configured to dispense medicament from the needle and wherein the actuation member is configured to release the dispensing mechanism when the actuation member is in the second position.

The medicament delivery device may further comprise a container containing the medicament.

According to another aspect of the present disclosure there is provided a method of preparing a medicament delivery device for use prior to dispensing medicament from the device, the method comprising removing a cap from the medicament delivery device, wherein the cap is configured to prevent movement of the actuation member from the first position to the second position when the cap is attached to the medicament delivery device.

According to another aspect of the present disclosure there is provided a method of using a medicament delivery device, the method comprising removing a cap from the medicament delivery device, wherein the cap is configured to prevent movement of the actuation member from the first position to the second position when the cap is attached to the medicament delivery device, and moving an actuation member for dispensing medicament from the medicament delivery device.

The portion may be a proximally-extending portion.

The needle may be for protruding from the distal end of the body for injecting medicament. The needle may be for protruding from the distal end of the medicament delivery device for injecting medicament. The needle may be configured to protrude from the distal end of the body. The needle may have a distal free end for injecting medicament.

The cap may be removably attached to the body. When the cap is attached to the body then the cap may cover the distal end of the body for preventing access to the needle.

The cap may be for preventing access to the needle.

The portion may be configured to prevent the actuation member from moving relative to the body.

The protrusion may engage the radially-extending surface when the actuation member is in the first position.

The portion may engage the axially-extending component on the opposite side of the axially-extending component to the protrusion to prevent distal movement of the actuation member to the second position.

The radially-extending surface may engage the axially-extending component when the actuation member is in the first position.

The medicament delivery device may comprise an axially-extending component, wherein the portion is configured to engage the axially-extending component to prevent movement of the actuation member from the first position to the second position.

The portion may engage the axially-extending component when the cap is attached to the body and the actuation member is in the first position.

The proximally-extending portion may be a proximally-extending arm.

The axially-extending component may be an axially-extending arm.

The cap may be configured to prevent the actuation member from moving relative to the body when the cap is attached to the body. The proximally-extending portion may be configured to prevent the actuation member from moving relative to the body when the cap is attached to the body.

The distal-facing surface may be orthogonal to the longitudinal axis of the medicament delivery device.

The portion may engage the axially-extending component on the opposite side of the axially-extending component to the radially-extending surface.

A proximal-facing surface on the portion may be configured to abut the distal facing surface of the axially-extending component.

The proximal-facing surface may be orthogonal to the longitudinal axis of the medicament delivery device.

The axially-extending component may be an axially-extending arm. In some embodiments, the distal-facing surface is provided on the free end of the axially-extending arm.

The distally-extending portion may be an axially-extending arm. In some embodiments, the proximal-facing surface is provided on the free end of the axially-extending arm.

The actuation member may comprise the axially-extending component.

The axially-extending component may comprise a sleeve. The distal-facing surface may be provided on the free end of the sleeve.

The portion may comprise a sleeve. The proximal-facing surface may be provided on the free end of the sleeve.

The cap may comprise a third axially-extending arm, and the actuation member may comprise a fourth axially-extending arm, wherein the third axially-extending arm is configured to abut a distal-facing surface on the fourth axially-extending arm to prevent the actuation member from moving to the second position.

The button may be provided at the proximal end of the medicament delivery device.

The actuation member may be distally movable relative to the body from the first position to the second position.

Moving the actuation member from the first position to the second position may comprise pressing the actuation member.

The dispensing mechanism may be configured to cause the needle to move distally from a needle pre-use position to a needle injection position in which the needle protrudes from the distal end of the body when the dispensing mechanism is released.

According to another aspect of the present disclosure there is provided a medicament delivery device for injecting medicament, wherein the medicament delivery device comprises a body having a proximal end and a distal end; a needle for injecting medicament; an actuation member which is movable relative to the body from a first position to a second position for dispensing medicament from the needle; and a cap which is removably attached to the body. When the cap is attached to the body then the cap may cover the distal end of the body for preventing access to the needle. The cap may comprise a proximally-extending portion configured to prevent movement of the actuation member from the first position to the second position when the cap is attached to the body.

The medicament delivery device may comprise an axially-extending component and wherein the proximally-extending portion is configured to engage the axially-extending component to prevent movement of the actuation member from the first position to the second position.

The proximally-extending portion may engage the axially-extending component when the cap is attached to the body and the actuation member is in the first position.

The medicament delivery device may comprise a radially-extending surface which is configured to engage the axially-extending component and wherein the proximally-extending portion and the radially-extending surface cooperate to prevent distal movement of the actuation member.

The radially-extending surface may engages the axially-extending component when the actuation member is in the first position.

The axially-extending component may comprise a protrusion which extends radially from the axially-extending component, and wherein the radially-extending surface is configured to engage the protrusion.

The proximally-extending portion may engage the axially-extending component on the opposite side of the axially-extending component to the radially-extending surface.

The proximally-extending portion may abut the distal-facing surface when the cap is attached to the body and the actuation member is in the first position.

The proximally-extending portion may be a proximally-extending arm.

The axially-extending component may be an axially-extending arm.

The proximally-extending portion may be configured to prevent the actuation member from moving relative to the body when the cap is attached to the body.

According to another aspect of the present disclosure there is provided a method of preparing a medicament delivery device for use prior to dispensing medicament from the device, the method comprising removing a cap from the medicament delivery device, wherein the cap comprises a proximally-extending portion configured to prevent movement of the actuation member from the first position to the second position when the cap is attached to the body.

According to another aspect of the present disclosure there is provided a method of using a medicament delivery device, the method comprising removing a cap from the medicament delivery device, wherein the cap comprises a proximally-extending portion configured to prevent movement of the actuation member from the first position to the second position when the cap is attached to the body, and moving an actuation member for dispensing medicament from the medicament delivery device.

According to another aspect of the present disclosure there is provided a method of manufacturing or assembling a medicament delivery device, wherein the medicament delivery device is defined in claim 1. Further optional features of the medicament delivery device are described and/or contemplated here.

According to another aspect of the present disclosure there is provided a method of manufacturing or assembling a medicament delivery device, wherein the medicament delivery device has the features of any of the medicament delivery devices described and/or contemplated herein.

### BRIEF DESCRIPTION OF THE DRAWINGS

Embodiments will now be described, by way of example only, with reference to the accompanying drawings, in which:

FIG. 1A is a schematic view of a medicament delivery device with a cap attached;

FIG. 1B is a schematic view of the medicament delivery device of FIG. 1A with the cap removed;

FIG. 2A is a schematic view of a medicament delivery device prior to use (i.e. in a pre-use configuration);

FIG. 2B is a schematic view of the device of FIG. 2A with the cap removed;

FIG. 2C is a schematic view of the device of FIG. 2A showing the device placed at an injection site;

FIG. 2D is a schematic view of the device of FIG. 2A with the button having been pressed to release the dispensing mechanism;

FIG. 2E is a schematic view of the device of FIG. 2A with the button having been pressed to release the dispensing mechanism;

FIG. 2F is a schematic view of the device of FIG. 2A showing the needle having retracted within the device after a dose has been delivered;

FIG. 2G is a schematic view of the device of FIG. 2A showing the device removed from the injection site after the needle has retracted within the device after delivery of the medicament;

FIG. 3A is a schematic view of parts of a medicament delivery device according to an embodiment of the present invention;

FIG. 3B is a schematic view of the device of FIG. 3A with the cap being removed and the actuation member in the first position;

FIG. 3C is a schematic view of the device of FIG. 3A with the cap removed and the actuation member in the second position;

FIG. 4 is a schematic view of parts of a medicament delivery device according to another embodiment of the present disclosure.

FIG. 5A is a schematic view of parts of a medicament delivery device according to an embodiment of the present disclosure;

FIG. 5B is a schematic view of the device of FIG. 5A with the cap removed and the actuation member in the first position;

FIG. 5C is a schematic view of the device of FIG. 5A with the cap removed and device positioned on an injection site;

FIG. 6 is a schematic view of parts of a medicament delivery device according to another embodiment of the present disclosure;

FIG. 7A is a schematic view of parts of a medicament delivery device according to another embodiment of the present disclosure;

FIG. 7B is a schematic view of the device of FIG. 7A with the cap moved distally;

FIG. 7C is a schematic view of the device of FIG. 7A with the cap moved further distally;

FIG. 7D is a schematic view of the device of FIG. 7A with the cap fully removed from the medicament delivery device;

FIG. 8A is a schematic view of parts of a medicament delivery device according to another embodiment of the present disclosure;

FIG. 8B is a schematic view of the medicament delivery device of FIG. 8A with the cap removed from the medicament delivery device; and

FIG. 8C is a schematic view of the medicament delivery device of FIG. 8A with the actuation member in the second position.

### DETAILED DESCRIPTION

A drug delivery device, as described herein, may be configured to inject a medicament into a patient. For example, delivery could be sub-cutaneous, intra-muscular, or intravenous. Such a device could be operated by a patient or care-giver, such as a nurse or physician, and can include various types of safety syringe, pen-injector, or auto-injector. The device can include a cartridge-based system that requires piercing a sealed ampule before use. Volumes of medicament delivered with these various devices can range from about 0.5 ml to about 2 ml. Yet another device can include a large volume device (“LVD”) or patch pump, configured to adhere to a patient's skin for a period of time (e.g., about 5, 15, 30, 60, or 120 minutes) to deliver a “large” volume of medicament (typically about 2 ml to about 10 ml).

In combination with a specific medicament, the presently described devices may also be customized in order to operate within required specifications. For example, the device may be customized to inject a medicament within a certain time period (e.g., about 3 to about 20 seconds for auto-injectors, and about 10 minutes to about 60 minutes for an LVD). Other specifications can include a low or minimal level of discomfort, or to certain conditions related to human factors, shelf-life, expiry, biocompatibility, environmental considerations, etc. Such variations can arise due to various factors, such as a drug ranging in viscosity from about 3 cP to about 50 cP. Consequently, a drug delivery device will often include a hollow needle ranging from about 25 to about 31 Gauge in size. Common sizes are 27 and 29 Gauge.

The delivery devices described herein can also include one or more automated functions. For example, one or more of needle insertion, medicament injection, and needle retraction can be automated. Energy for one or more automation steps can be provided by one or more energy sources. Energy sources can include, for example, mechanical, pneumatic, chemical, or electrical energy. For example, mechanical energy sources can include springs, levers, elastomers, or other mechanical mechanisms to store or release energy. One or more energy sources can be combined into a single device. Devices can further include gears, valves, or other mechanisms to convert energy into movement of one or more components of a device.

The one or more automated functions of an auto-injector may each be activated via an activation mechanism. Such an activation mechanism can include one or more of a button, a lever, a needle sleeve, or other activation component. Activation of an automated function may be a one-step or multi-step process. That is, a user may need to activate one or more activation components in order to cause the automated function. For example, in a one-step process, a user may depress a needle sleeve against their body in order to cause injection of a medicament. Other devices may require a multi-step activation of an automated function. For example, a user may be required to depress a button and retract a needle shield in order to cause injection.

In addition, activation of one automated function may activate one or more subsequent automated functions, thereby forming an activation sequence. For example, activation of a first automated function may activate at least two of needle insertion, medicament injection, and needle retraction. Some devices may also require a specific sequence of steps to cause the one or more automated functions to occur. Other devices may operate with a sequence of independent steps.

Some delivery devices can include one or more functions of a safety syringe, pen-injector, or auto-injector. For example, a delivery device could include a mechanical energy source configured to automatically inject a medicament (as typically found in an auto-injector) and a dose setting mechanism (as typically found in a pen-injector).

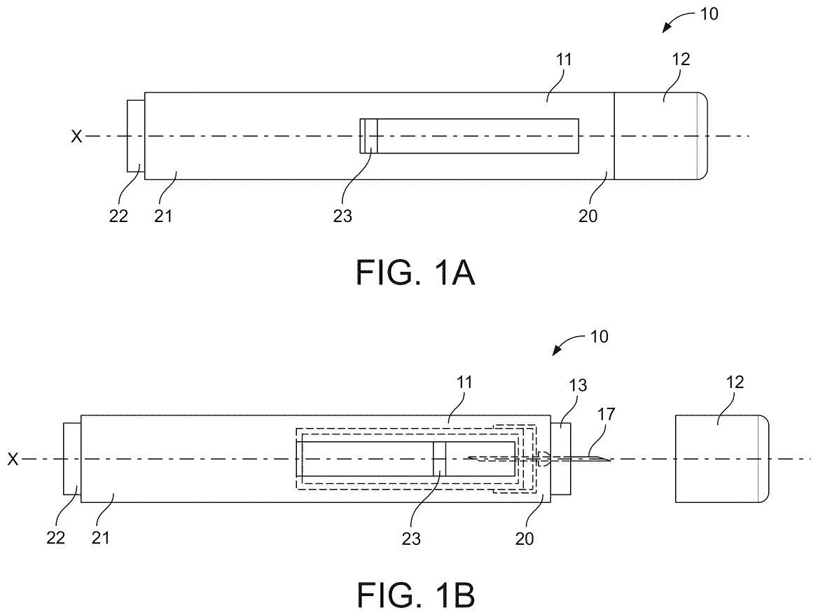

According to some embodiments of the present disclosure, an exemplary drug delivery device 10 is shown in FIGS. 1A & 1B. The device 10, as described above, is configured to inject a medicament into a patient's body. The device 10 includes a housing 11 which typically contains a reservoir containing the medicament to be injected (e.g., a syringe) and the components required to facilitate one or more steps of the delivery process. The device 10 can also include a cap assembly 12 that can be detachably mounted to the housing 11. A user typically removes the cap 12 from the housing 11 before the device 10 is operated.

As shown, the housing 11 is substantially cylindrical and has a substantially constant diameter along the longitudinal axis X. The housing 11 has a distal region 20 and a proximal region 21. The term “distal” refers to a location that is relatively closer to a site of injection, and the term “proximal” refers to a location that is relatively further away from the injection site.

The device 10 can also include a needle sleeve 13 coupled to the housing 11 to permit movement of the sleeve 13 relative to the housing 11. For example, the sleeve 13 can move in a longitudinal direction parallel to longitudinal axis X. Specifically, movement of the sleeve 13 in a proximal direction can permit a needle 17 to extend from the distal region 20 of the housing 11.

Insertion of the needle 17 can occur via several mechanisms. For example, the needle 17 may be fixedly located relative to the housing 11 and initially be located within an extended needle sleeve 13. Proximal movement of the sleeve 13 by placing a distal end of the sleeve 13 against a patient's body and moving the housing 11 in a distal direction will uncover the distal end of the needle 17. Such relative movement allows the distal end of the needle 17 to extend into the patient's body. Such insertion is termed “manual” insertion as the needle 17 is manually inserted via the patient's manual movement of the housing 11 relative to the sleeve 13.

Another form of insertion is “automated,” whereby the needle 17 moves relative to housing 11. Such insertion can be triggered by movement of the sleeve 13 or by another form of activation, such as a button 22. As shown in FIGS. 1A & 1B, the button 22 is located at a proximal end of the housing 11. However, in other embodiments, the button 22 could be located on a side of the housing 11.

Other manual or automated features can include drug injection or needle retraction, or both. Injection is the process by which a bung or piston 23 is moved from a proximal location within a syringe (not shown) to a more distal location within the syringe in order to force a medicament from the syringe through the needle 17. In some embodiments, a drive spring (not shown) is under compression before the device 10 is activated. A proximal end of the drive spring can be fixed within the proximal region 21 of the housing 11, and a distal end of the drive spring can be configured to apply a compressive force to a proximal surface of the piston 23. Following activation, at least part of the energy stored in the drive spring can be applied to the proximal surface of the piston 23. This compressive force can act on the piston 23 to move it in a distal direction. Such distal movement acts to compress the liquid medicament within the syringe, forcing it out of the needle 17.

Following injection, the needle 17 can be retracted within the sleeve 13 or the housing 11. Retraction can occur when the sleeve 13 moves distally as a user removes the device 10 from a patient's body. This can occur as the needle 17 remains fixedly located relative to the housing 11. Once a distal end of the sleeve 13 has moved past a distal end of the needle 17, and the needle 17 is covered, the sleeve 13 can be locked. Such locking can include locking any proximal movement of the sleeve 13 relative to the housing 11.

Another form of needle retraction can occur if the needle 17 is moved relative to the housing 11. Such movement can occur if the syringe within the housing 11 is moved in a proximal direction relative to the housing 11. This proximal movement can be achieved by using a retraction spring (not shown), located in the distal region 20. A compressed retraction spring, when activated, can supply sufficient force to the syringe to move it in a proximal direction. Following sufficient retraction, any relative movement between the needle 17 and the housing 11 can be locked with a locking mechanism. In addition, the button 22 or other components of device 10 can be locked as required.

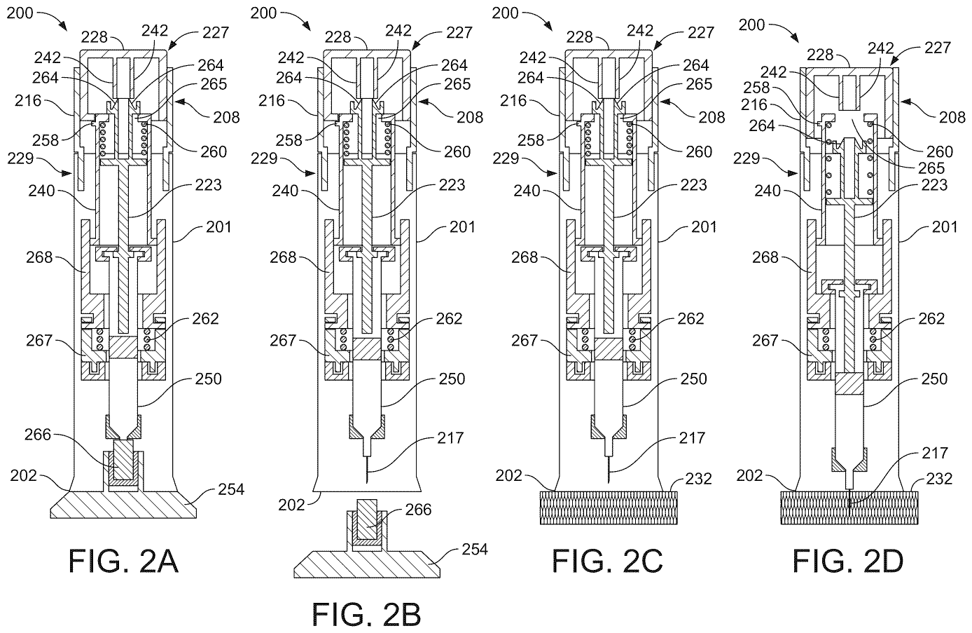

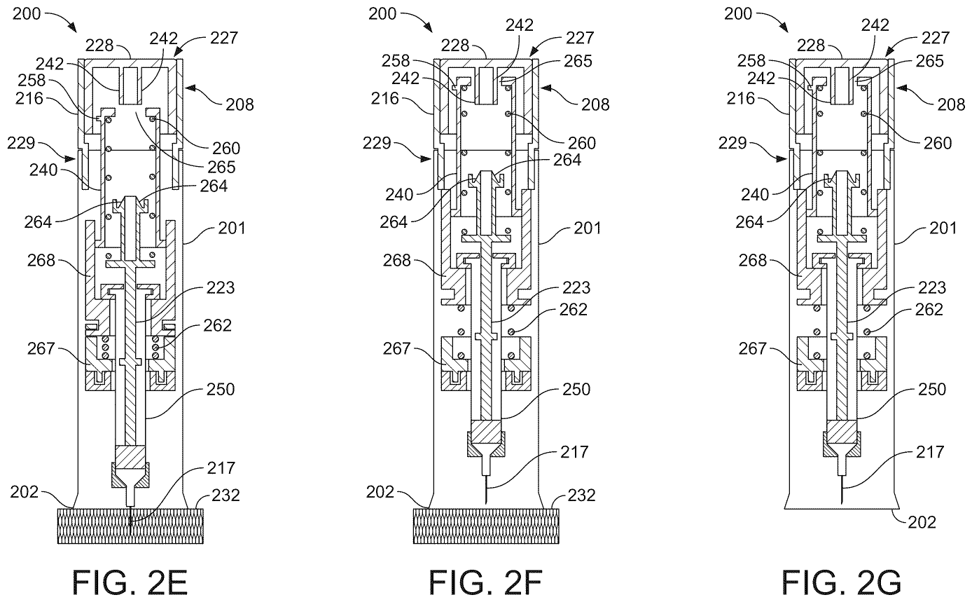

FIGS. 2A to 2G show the sequential steps of operating a medicament delivery device 200. The medicament delivery device 200 is an autoinjector.

The device 200 comprises a body 201, a syringe 250 having a needle 217 and an axially moveable plunger 223 for dispensing medicament from the syringe 250. The device comprises a cap 254 which is removably attached to the body 201 and covers a distal end 202 of the body 201 for preventing access to the needle 217. The device has a needle shield 266 that covers the needle 217 before use. The needle shield 266 is attached to the cap 254.

The medicament delivery device 200 has a dispensing mechanism 229. The medicament delivery device 200 has an actuation member 227 which is configured to release the dispensing mechanism 229. The actuation member 227 is configured to engage the dispensing mechanism 229 to release the dispensing mechanism 229.

The dispensing mechanism 229 is configured to cause the needle 217 to move distally from a needle pre-use position, in which the needle 217 is recessed within the body 201, to an injection position in which the needle 217 protrudes from the distal end 202 of the body 201 when the dispensing mechanism 229 is released.

The dispensing mechanism 229 is configured to dispense the medicament from the needle 217 when the needle 217 is in the injection position.

As shown in FIGS. 2B-2C, in order to deliver a dose of medicament to an injection site, the cap 254 is removed (FIG. 2B) and the device is placed at an injection site 232 (FIG. 2C).

The actuation member 227 comprises a button 228 and is prevented from being depressed by a stop 258. The stop is provided on the spring guide 240, for example.

The device has a locking member 208 in the form of a lock ring 216 which is rotatable by a user about a longitudinal axis of the device. The actuation member 227 is keyed to the lock ring 216 so that the actuation member 227 rotates with the lock ring 216. The lock ring 216 is rotatable from a pre-use position, in which distal movement of the button 228 is prevented, to a use position in which distal movement of the button 228 is permitted.

When the lock ring 216 is in the pre-use position then the stop 258 engages the button 228 to prevent the button 228 from being depressed.

In order to allow the button 228 to be depressed, the lock ring 216 is rotated about the longitudinal axis of the device from the pre-use position to the use position. The rotation of the lock ring 216 also rotates the actuation member 227 to a position in which the stop 258 no longer prevents the button 228 from being depressed as shown, for example, in FIG. 2C.

Turning now to FIG. 2D, the user then presses the button 228 to release the dispensing mechanism 229 for dispensing medicament from the device. The dispensing mechanism 229 has a plunger 223 and a bias in the form of a compression spring 260. The plunger 223 is biased distally by the spring 260.

The dispensing mechanism 229 is at least partially housed within the spring guide 240. The plunger 223 has a release member which has proximally-extending clips 264. The spring 260 is retained in the compressed position by virtue of the clips 264 which protrude through a proximal opening 265 in the spring guide 240. The clips 264 engage the spring guide 240 for maintaining the plunger 223 in a proximal position.

The actuation member 227 has a firing member comprising a pair of protrusions 242 which engage with the clips 264 when the button 228 is depressed to flex the clips 264 radially inwardly thereby allowing the clips 264 to move distally through the proximal opening 265 to release the spring 260.

When the dispensing mechanism 229 is released, then the syringe 250 is released for distal axial movement towards the injection site 232 such that the needle 217 moves from the needle pre-use retracted position to an exposed (or “uncovered” or “injection”) position for delivering medicament to the injection site 232 under the biasing force of the compression spring 260.

Depressing the button 228 releases the plunger 223 which, biased by the bias 260, moves along the syringe 250 towards the distal end of the device 200 to force medicament within the syringe 250 through the needle 217, thereby delivering a dose of medicament as shown, for example in FIG. 2E.

As shown in FIG. 2F, once the dose of medicament has been delivered, a medicament container bias 262, embodied by a further spring 262, then causes the needle 217 to move axially back to the retracted position, away from the injection site 232 in a proximal direction. The plunger 223 flexes a clip (not shown) on a first collar 267 which allows the first collar 267 to rotate relative to the body 201 and relative to a second collar 268. The first collar 267 rotates from a first position in which the second collar 268 is axially coupled to the first collar 267, into a second position in which the second collar 268 is free to move axially relative to the first collar 267. For example, the second collar 268 may comprise a radially protruding coupling element configured to be received in or engage with a corresponding receiving portion of the first collar 267, such that rotating the first collar 267 from the first position into the second position causes the coupling element to be moved out from the receiving portion, to allow the second collar 268 to move axially relative to the first collar 267. Axial movement of the second collar 268 permits the needle 217 to be retracted.

As shown in FIG. 2G, the device 200 is then removed from the injection site 232, for disposal.

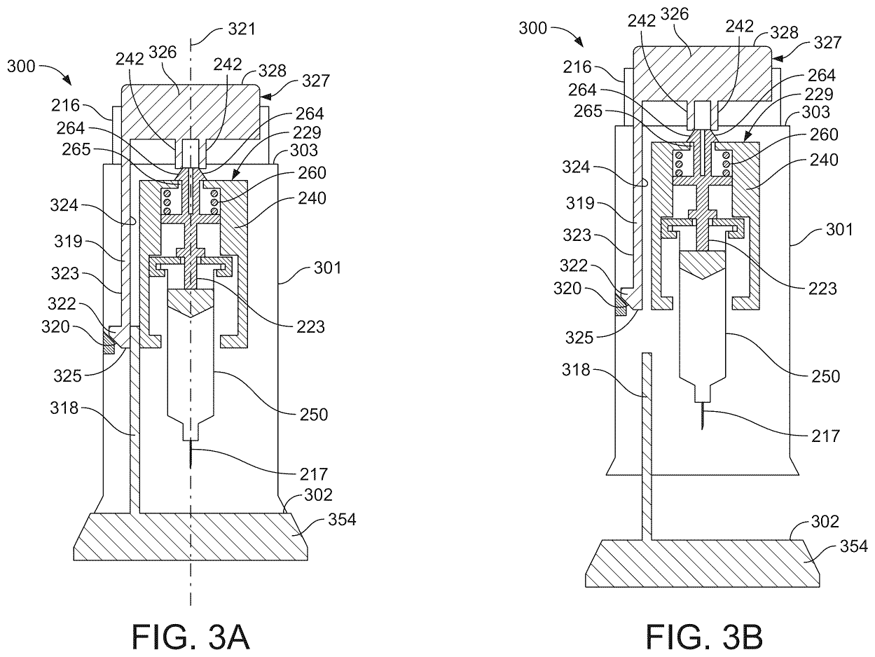

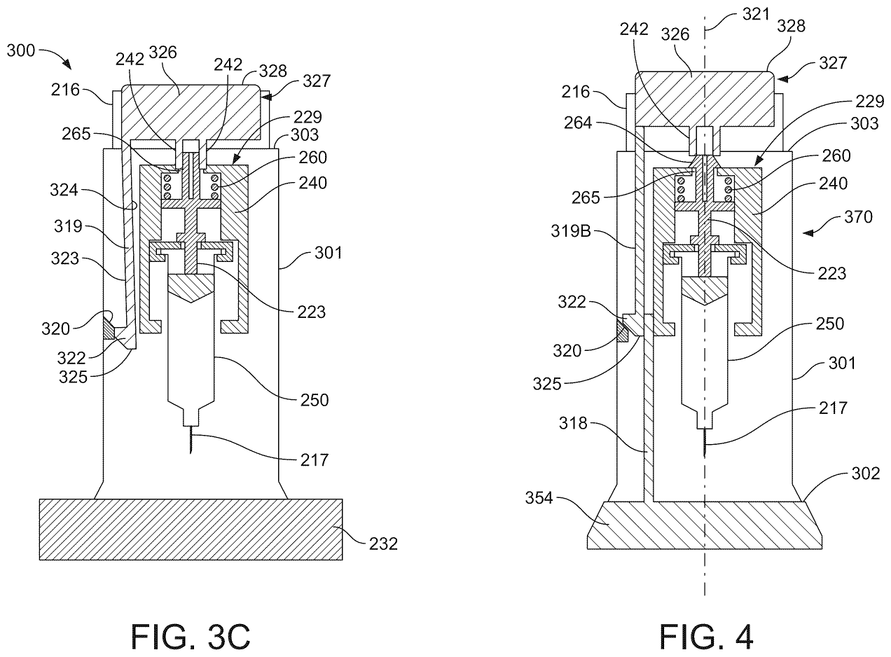

FIG. 3A to 3C are schematic views of parts of a medicament delivery device 300 in accordance with an embodiment of the disclosure.

The medicament delivery device 300 has a body 301 having a proximal end 303 and a distal end 302, a needle 217 for injecting medicament, and an actuation member 327 which is movable relative to the body 301 from a first position to a second position for dispensing medicament from the needle 217. FIGS. 3A and 3B show the actuation member 327 in an example first position. FIG. 3C shows the actuation member 327 in an example second position. The actuation member 327 is distally movable relative to the body 301 from the first position to the second position.

The needle 217 is for protruding from the distal end of the medicament delivery device for injecting medicament. The needle 217 is for protruding from the distal end 302 of the body for injecting medicament. The needle 217 is configured to protrude from the distal end of the body 301 when the actuation member is in the second position. The needle 217 has a distal free end for injecting medicament.

The needle 217 is part of a syringe 250 which contains the medicament. The syringe 250 can be seen as a container containing the medicament. The syringe 250 can be said to comprise the needle 217. Another embodiment may comprise a container which contains medicament, wherein the container is initially separated from the needle before the actuation member is moved from the first position to the second position. The container may be a cartridge of medicament. Movement of the actuation member from the first position to the second position may cause the proximal end of the needle to pierce the container.

The medicament delivery device 300 has a cap 354 which is removably attached to the body 301. When the cap 354 is attached to the body 301 then the cap 354 covers the distal end 302 of the body 301 for preventing access to the needle 217. The cap 354 may include one or more apertures therein.

In another embodiment the cap 354 is attached to another component of the medicament delivery device.

The cap 354 has a proximally-extending portion 318 which is configured to prevent movement of the actuation member 327 from the first position to the second position when the cap 354 is attached to the body 301. The proximally-extending portion 318 may extend into the body 301 when the cap 354 is attached to the body 301. The proximally-extending portion 318 may be an axially-extending arm but in another embodiment the proximally-extending portion 318 may be a sleeve, for example a circular sleeve.

The actuation member 327 has an axially-extending component 319. The actuation member 327 can be said to comprise the axially-extending component 319. The proximally-extending portion 318 engages the axially-extending component 319 when the cap 354 is attached to the body 301 and the actuation member is in the first position to prevent movement of the actuation member 327 from the first position to the second position. The axially-extending component 319 is movable relative to the body 301.

The proximally-extending portion 318 prevents the actuation member 327 from moving relative to the body 301.

In another embodiment, the proximally-extending portion 318 allows the actuation member 327 to move relative to the body provided that the proximally-extending portion 318 prevents the actuation member 327 from reaching the second position.

For example, there may be a gap between the proximally-extending portion 318 and the axially-extending component 319 when the actuation member 327 is in the first position. The proximally-extending portion 318 engages the axially-extending component 319 when the actuation member 327 moves from the first position towards the second position to prevent the actuation member 327 from reaching the second position when the cap 354 is attached to the body 301.

The medicament delivery device has a radially-extending surface 320 which engages the axially-extending component 319 when the actuation member 327 is in the first position. The radially-extending surface 320 is a planar surface at an acute angle to the longitudinal axis 321 of the medicament delivery device. Alternatively or additionally the radially extending surface 320 may comprise a curved surface. The radially-extending surface 320 is fixed relative to the body 301 for example the radially-extending surface may be provided on an inner sleeve or another component of the medicament delivery device 300. In another embodiment the radially-extending surface 320 is provided on the body 301.

The axially-extending component 319 has a protrusion 322 which extends radially from an exterior side 323 of the axially extending component 319. The exterior side 323 is the side which faces radially outwards from the longitudinal axis 321. The protrusion 322 engages the radially-extending surface 320 when the actuation member 327 is in the first position. The protrusion 322 is located proximally of the radially-extending surface 320 when the actuation member 327 is in the first position for preventing distal movement of the actuation member 327 from the first position to the second position.

In another embodiment there is a gap between the protrusion 322 and the radially-extending surface 320 when the actuation member 327 is in the first position. The protrusion 322 engages the radially-extending surface 320 when the actuation member 327 moves from the first position towards the second position to prevent the actuation member 327 from reaching the second position when the cap 354 is attached to the body 301.

The proximally-extending portion 318 engages the axially-extending component 319 on the opposite side 324 of the axially-extending component 319 to the radially-extending surface 320 for maintaining the engagement between the axially-extending component 319 and the radially-extending surface 320. If a protrusion 322 is present then the proximally-extending portion 318 engages the axially-extending component 319 on the opposite side 324 of the axially-extending component 319 to the protrusion. The opposite side 324 in this example is an interior side 324 which faces radially inwards to the longitudinal axis 321.

The axially-extending component 319 is flexible. The proximally-extending portion 318 and the radially-extending surface 320 cooperate to prevent distal movement of the actuation member 327 from the first position to the second position. When the cap 354 is attached to the body 301 then the protrusion 322 engages the radially-extending surface 320 and the proximally-extending portion 318 engages the axially-extending component 319. The proximally-extending portion 318 prevents the axially-extending component 319 from being deflected radially by the radially-extending surface 320. The proximally-extending portion 318 and the axially-extending component 319 are disengaged when the cap 354 is removed and the axially-extending component 319 is permitted to be deflected by the radially-extending surface 320 as the actuation member 327 moves distally.

When the actuation member 327 moves from the first position to the second position then the axially-extending component 319 is deflected radially by the engagement of the axially-extending component 319 and the radially-extending surface 320. If the axially-extending component 319 has a protrusion then when the actuation member 327 moves from the first position to the second position then the axially-extending component 319 is deflected radially by the engagement of the protrusion 322 and the radially-extending surface 320.

The protrusion 322 is located at or towards the free end 325 of the axially-extending component 319. In another embodiment the protrusion 322 is located proximally of the free end 325 of the axially-extending component 319.

In another embodiment the axially-extending component 319 does not have a protrusion and instead a surface on the side of the axially-extending component 319 engages the radially-extending surface 320. This may be, for example, in embodiments in which there is limited axial travel of the actuation member 327 from the first position to the second position.

In the embodiment shown in FIGS. 3A to 3C, the proximally-extending portion 318 engages the axially-extending component 319 on an interior side 324 of the axially-extending component 319. The axially-extending component 319 flexes radially inwards when the actuation member 327 moves from the first position to the second position. However in another embodiment (not shown) the proximally-extending portion 318 engages the axially-extending component 319 on the exterior side 323 of the axially-extending component 319 and the protrusion 322 extends radially from the interior side 324 of the axially-extending component 319. The axially-extending component 319 then flexes radially outwards when the actuation member 327 moves from the first position to the second position.

The proximally-extending portion 318 is a first axially-extending arm. The axially-extending component 319 is a second axially-extending arm.

The first axially-extending arm 318 and the second axially-extending arm 319 form a pair of arms which interact to prevent the actuation member 327 from moving to the second position when the cap is attached to the body. In another embodiment, the medicament delivery device may have multiple pairs of arms, for example two or three pairs of arms, which interact in the same way as described for the first axially-extending arm 318 and the second axially-extending arm 319 to prevent the actuation member 327 from moving to the second position when the cap 354 is attached to the medicament delivery device. Each of the pairs of arms may be spaced around the circumference of the device, for example the pair of arms may be equally spaced around the circumference.

In another embodiment the proximally-extending portion 318 comprises a sleeve instead of the first axially-extending arm 318. The sleeve may be a cylindrical sleeve, for example. The sleeve operates in the same way as described and/or contemplated herein in relation to the first axially-extending arm 318.

Explicitly, the sleeve engages the second axially extending arm 319 when the cap 354 is attached to the body 301 and the actuation member 327 is in the first position.

In another embodiment there is a gap between the sleeve and the second axially-extending arm 319 when cap 354 is attached to the body and the actuation member 327 is in the first position. The sleeve engages the second axially-extending arm 319 when the actuation member 327 moves from the first position towards the second position to prevent the actuation member 327 from reaching the second position when the cap 354 is attached to the body 301.

The sleeve and the radially-extending surface 320 cooperate to prevent distal movement of the actuation member 327. The sleeve is configured to engage the second axially-extending arm 319 on the opposite side 324 of the second axially-extending arm 319 to the radially-extending surface 320 to prevent distal movement of the actuation member 327.

The actuation member 327 comprises a button 326 which is configured to be pressed to move the actuation member 327 from the first position to the second position. The button 326 is configured to be pressed to move the actuation member 327 from the first position to the second position. The button 326 may be integrally formed with the axially-extending component 319 or the axially-extending component 319 may be formed separately to the button 326 and connected to the button 326.

The button 326 is provided at the proximal end 328 of the medicament delivery device. In another embodiment (not shown) the button is provided on a lateral side of the medicament delivery device.

The medicament delivery device 300 has a lock ring 216 which is rotatable from a pre-use position, in which movement of the actuation member 327 from the first position to the second position is prevented, to a use position in which movement of the actuation member 327 from the first position to the second position is permitted.

The lock ring 216 and actuation member 327 may have corresponding features to those described and/or contemplated in relation to the corresponding components of the medicament delivery device 200.

In another embodiment the lock ring 216 is not present in the medicament delivery device.

In use, the cap 354 is removed from the medicament delivery device, for example from the body, by a user, for example. In some embodiments, the lock ring 216 (if present) is then rotated from the pre-use position to the use position. The button 326 is then pressed, for example by the user, when the medicament delivery device 300 is held against an injection site 232 to dispense the medicament via the needle 217.

The proximally-extending portion 318 prevents the user from accidentally dispensing the medicament from the needle 217 before the cap 354 has been removed from the device.

The medicament delivery device 300 has a dispensing mechanism 229 which is configured to dispense medicament from the needle 217. The actuation member 327 is configured to release the dispensing mechanism 229, for example by engaging the dispensing mechanism, when the actuation member 327 is in the second position.

The medicament delivery devices described herein may have some or all of the features as described in relation to the medicament delivery device 200.

The dispensing mechanism 229 may have the some or all of the features as described and/or contemplated in relation to FIGS. 2A to 2G.

In another embodiment, the dispensing mechanism may have alternative or additional features to those described and/or contemplated in relation to FIGS. 2A to 2G. The dispensing mechanism may have features as described and/or contemplated herein, for example in relation to FIGS. 1A and 1B.

The dispensing mechanism provides one or more automated functions. For example, one or more of needle insertion, medicament injection, and needle retraction can be automated. Energy for one or more automation steps can be provided by one or more energy sources. Energy sources can include, for example, mechanical, pneumatic, chemical, or electrical energy. For example, mechanical energy sources can include springs, levers, elastomers, or other mechanical mechanisms to store or release energy. One or more energy sources can be combined into a single device. Devices can further include gears, valves, or other mechanisms to convert energy into movement of one or more components of a device.

The one or more automated functions may each be activated via an activation mechanism. Such an activation mechanism can include one or more of a button, a lever, a needle sleeve, or other activation component. Activation of an automated function may be a one-step or multi-step process. That is, a user may need to activate one or more activation components in order to cause the automated function. For example, in a one-step process, a user may depress a needle sleeve against their body in order to cause injection of a medicament. Other devices may require a multi-step activation of an automated function. For example, a user may be required to depress a button and retract a needle shield in order to cause injection.

In addition, activation of one automated function may activate one or more subsequent automated functions, thereby forming an activation sequence. For example, activation of a first automated function may activate at least two of needle insertion, medicament injection, and needle retraction. Some devices may also require a specific sequence of steps to cause the one or more automated functions to occur. Other devices may operate with a sequence of independent steps.

Some delivery devices can include one or more functions of a safety syringe, pen-injector, or auto-injector. For example, a delivery device could include a mechanical energy source configured to automatically inject a medicament (as typically found in an auto-injector) and a dose setting mechanism (as typically found in a pen-injector).

The medicament delivery device can include various types of safety syringe, pen-injector, or auto-injector. The device can include a cartridge-based system that requires piercing a sealed ampule before use.

Distal movement of the actuation member may cause automatic dispensing of the medicament from the device and/or distal movement of the actuation member may cause the distal movement of the needle from a needle pre-use position to a needle injection position. The dispensing mechanism may be configured to dispense medicament from the needle when the dispensing mechanism is released.

In the needle pre-use position the needle may be flush with the distal end of the body or the needle may be recessed within the body. In another embodiment the needle may be fixed in position relative to the body.

In another device, different features may be provided to prevent the actuation member from moving distally. For example, the stop may be provided on another component of the medicament delivery device. In another device a lock ring 216 is not present.

A method of using the medicament delivery device 300 comprises removing the cap 354 from the medicament delivery device, for example from the body 301. The medicament delivery device may have any of the features described and/or contemplated herein. In some embodiments, the method comprises rotating the lock ring from a pre-use position to a use position. The method may further comprise moving the actuation member from the first position to the second position. Moving the actuation member from the first position to the second position may comprise a user pressing the button 326.

FIG. 4 is a schematic view of parts of a medicament delivery device 370 in accordance with an embodiment of the disclosure. The medicament delivery device 370 has corresponding features as described and/or contemplated herein in relation to the medicament delivery device 300. The same reference numbers are used to annotate corresponding features of the medicament delivery device 370 as for the medicament delivery device 300.

In the medicament delivery device 370, the axially-extending component is an axially-extending arm 319B which is a separate component to the actuation member 327. The axially-extending arm 319B of the medicament delivery device 370 otherwise has the same features and functionality as described and/or contemplated in relation to the axially-extending component 319 of the medicament delivery device 300.

In another embodiment the axially-extending component may be a sleeve instead of the axially-extending arm 319B.

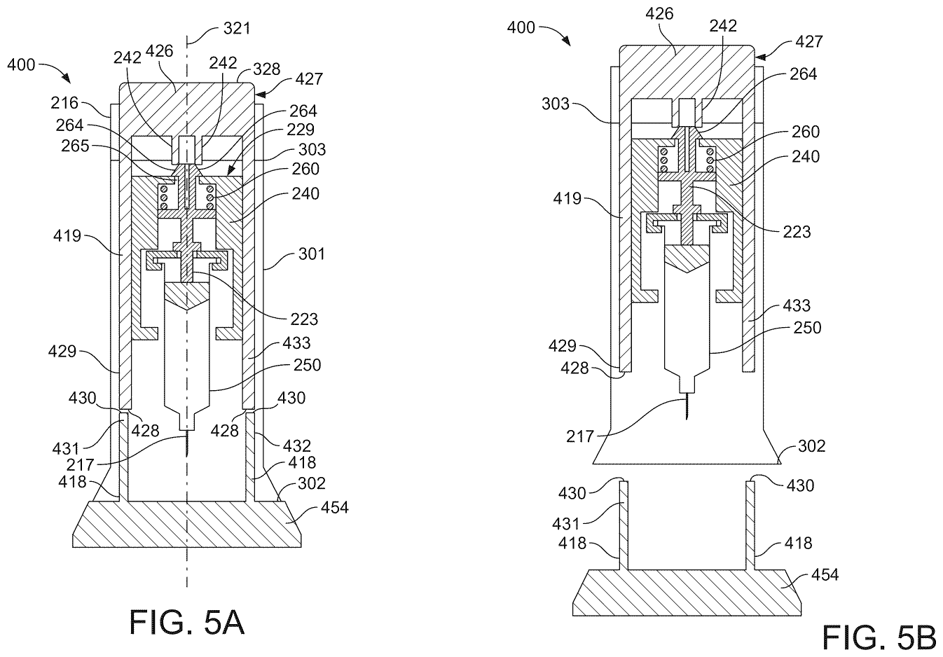

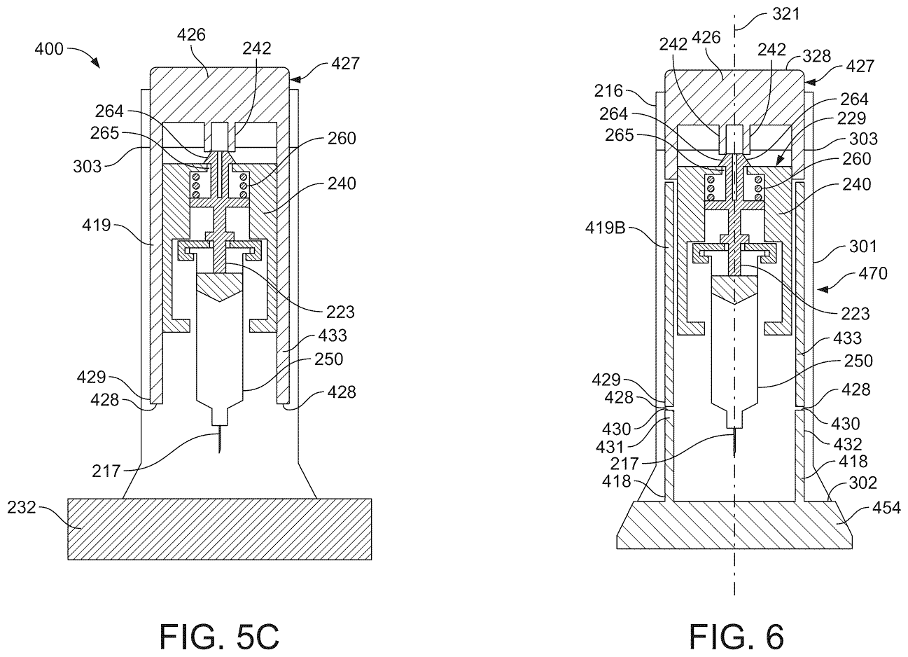

FIG. 5A to 5C are schematic views of parts of a medicament delivery device 400 in accordance with an embodiment of the disclosure. The medicament delivery device 400 has corresponding features as described and/or contemplated herein in relation to the medicament delivery device 300 and/or the medicament delivery device 200. The same reference numbers are used to annotate corresponding features of the medicament delivery device 400 as for the medicament delivery device 300 and/or the medicament delivery device 200.

The medicament delivery device 400 has an actuation member 427 which is movable relative to the body 301 from a first position to a second position for dispensing medicament from the needle 217. FIGS. 5A to 5C show the actuation member 427 in an example first position. The actuation member 427 is distally movable relative to the body 301 from the first position to the second position.

The medicament delivery device 400 has a cap 454 which is removably attached to the body 301. When the cap 454 is attached to the body 301 then the cap 454 covers the distal end 302 of the body 301 for preventing access to the needle 217.

In an embodiment the cap 454 is removably attached to another component of the medicament delivery device 400.

The cap 454 has a proximally-extending portion which is configured to prevent movement of the actuation member 427 from the first position to the second position when the cap 354 is attached to the body 301. The proximally-extending portion 418 may extend into the body 301 when the cap 354 is attached to the body 301.

The actuation member 427 has an axially-extending component 419. The actuation member 427 can be said to comprise the axially-extending component 419. The proximally-extending portion 418 engages the axially extending component 419 when the cap 454 is attached to the body 301 and the actuation member 427 is in the first position to prevent movement of the actuation member 427 from the first position to the second position. The axially-extending component 419 is movable relative to the body 301.

The proximally-extending portion 418 is configured to abut a distal-facing surface 428 on the axially-extending component 419 to prevent the actuation member 427 from moving to the second position. The distal-facing surface 428 may be on the free end 429 of the axially-extending component 419 or located proximally of the free end 429. The proximally-extending portion 418 abuts the distal-facing surface 429 when the cap 354 is attached to the body 301 and the actuation member 427 is in the first position. The actuation member 427 is therefore prevented from moving distally relative to the body 301.

The proximally-extending portion 418, which may be in the form of an axially-extending arm 418 or a sleeve, for example, prevents the actuation member 427 from moving relative to the body 301.

In another embodiment, the proximally-extending portion 418 allows the actuation member 427 to move relative to the body 301 provided that the proximally-extending portion 418 prevents the actuation member 427 from reaching the second position.

For example, there may be a gap between the proximally-extending portion 418 and the axially-extending component 419 when the actuation member 427 is in the first position. The axially-extending component 419 engages the proximally-extending portion 418 when the actuation member 427 moves from the first position towards the second position. The engagement of the proximally-extending portion 418 and the axially-extending component 419 prevents the actuation member 427 from reaching the second position.

The distal-facing surface 428 is orthogonal to the longitudinal axis 321 of the medicament delivery device 400. In another embodiment, the distal-facing surface 428 may be curved or at an acute angle to the longitudinal axis 321.

The proximally-extending portion 418 abuts the distal-facing surface 428 of the axially-extending component 419 via a proximal-facing surface 430 on the proximally-extending portion 418. The proximal-facing surface 430 may be on the free end 431 of the proximally-extending portion 418 or located proximally of the free end 431.

The proximal-facing surface 430 is orthogonal to the longitudinal axis 321 of the medicament delivery device. In another embodiment, the proximal-facing surface 430 may be curved or at an acute angle to the longitudinal axis 321.

The proximally-extending portion 418 is a first axially-extending arm. The axially-extending component 319 is a second axially-extending arm.

The first axially-extending arm 418 and the second axially-extending arm 419 form a pair of arms which interact to prevent the actuation member 427 from moving from the first position to the second position when the cap is attached to the body. The medicament delivery device 400 may have a single pair of arms or multiple pairs of arms, for example two or three pairs of arms, which interact in the same way as described for the first axially-extending arm 418 and the second axially-extending arm 419 to prevent the actuation member 427 from moving to the second position when the cap 354 is attached to the body 301. Each of the pairs of arms may be equally spaced around the circumference of the device.

For example, as shown the embodiment of FIGS. 5A to 5C, the cap 354 has a third axially-extending arm 432, and the actuation member has a fourth axially-extending arm 433 which have corresponding features as described and/or contemplated in relation to the first axially-extending arm 418 and the second axially-extending arm 419 respectively.

In another embodiment, the proximally-extending portion 418 comprises a sleeve instead of the first axially-extending arm 418. The sleeve may be a cylindrical sleeve, for example. The sleeve operates in the same way as described and/or contemplated herein in relation to the first axially-extending arm 418.

If the proximally-extending portion 418 is in the form of a sleeve then the sleeve is configured to abut the distal-facing surface 428 on the second axially-extending arm 419 to prevent the actuation member 427 from moving to the second position. The sleeve abuts the distal-facing surface 429 when the cap 354 is attached to the body 301 and the actuation member 427 is in the first position. In another embodiment there is a gap between the sleeve and the second axially-extending arm 419 when the actuation member 427 is in the first position. The second axially-extending arm 419 engages the sleeve when the actuation member 427 moves from the first position towards the second position to prevent the actuation member 427 from reaching the second position.

In another embodiment, the axially-extending component 419 comprises a sleeve. The sleeve may be a cylindrical sleeve, for example. The sleeve operates in the same way as described and/or contemplated herein in relation to the second axially-extending arm 419.

The proximally-extending portion 418 may be in the form of an axially-extending arm or sleeve, which is configured to abut a distal-facing surface on the axially-extending component 419 which is in the form of a sleeve to prevent the actuation member 427 from moving to the second position. The proximally-extending portion 418 may abut the sleeve when the actuation member 427 is in the first position or there may be a gap between the proximally-extending portion and the sleeve when the actuation member 427 is in the first position. The distal-facing surface of the sleeve may be orthogonal to the longitudinal axis 321.

The actuation member 427 has a button 426 which is configured to be pressed to move the actuation member 427 from the first position to the second position. The button 426 is configured to be pressed in the distal direction to move the actuation member 427 from the first position to the second position. The button 426 may be integrally formed with the axially-extending component 419 or the axially-extending component 419 may be formed separately to the button 426 and connected to the button 426.

The button 426 is provided at the proximal end 328 of the medicament delivery device. In another embodiment (not shown) the button 426 is provided on a lateral side of the medicament delivery device.

The medicament delivery device 400 has a dispensing mechanism 229 which is configured to dispense medicament from the needle 217. The actuation member 327 is configured to release the dispensing mechanism 229, for example by engaging the dispensing mechanism, when the actuation member 327 is in the second position.

As noted above, the dispensing mechanism 229 may have alternative or additional features to those described and/or contemplated in relation to FIGS. 2A to 2G. The dispensing mechanism may have features as described and/or contemplated herein, for example in relation to FIGS. 1A and 1B.

In another embodiment (not shown) the actuation member 427 does not have an axially extending component and instead the first proximally-extending portion 418, such as the first axially-extending arm 418 or sleeve, abuts a distal-facing surface of the button 426 to prevent the actuation member from moving to the second position.

In use, the cap 454 is removed from the medicament delivery device, for example from the body 301, by a user, for example. In some embodiments, the lock ring 216 (if present) is then rotated from the pre-use position to the use position. The button 426 is then pressed, for example by the user, when the medicament delivery device 400 is held against an injection site 232 to dispense the medicament via the needle 217.

The proximally-extending portion 418 prevents the user from accidentally dispensing the medicament from the needle 217 before the cap 454 has been removed from the device.

A method of using the medicament delivery device 400 comprises removing the cap 454 from the body 301. The medicament delivery device may have any of the features described and/or contemplated herein. In some embodiments, the method comprises rotating the lock ring 216 from a pre-use position to a use position. The method may further comprise moving the actuation member 427 from the first position to the second position. Moving the actuation member from the first position to the second position comprises a user pressing the button 426.

FIG. 6 is a schematic view of parts of a medicament delivery device 470 in accordance with an embodiment of the disclosure. The medicament delivery device 470 has corresponding features as described and/or contemplated herein in relation to the medicament delivery device 400. The same reference numbers are used to annotate corresponding features of the medicament delivery device 470 as for the medicament delivery device 400.

In the medicament delivery device 470, the axially-extending component 419B, which may be an axially-extending arm or a sleeve for example, is a separate component to the actuation member 427. The axially-extending component otherwise has the same features and functionality as described and/or contemplated in relation to the axially-extending component 419 of the medicament delivery device 400.

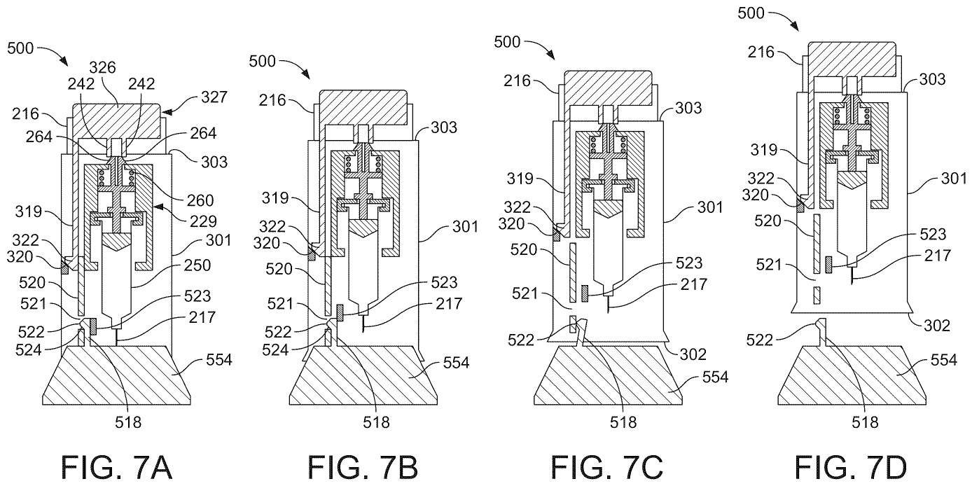

FIGS. 7A to 7D show a schematic view of parts of a medicament delivery device 500 in accordance with an embodiment of the disclosure. The medicament delivery device 500 has corresponding features as described and/or contemplated herein in relation to the medicament delivery device 300. The same reference numbers are used to annotate corresponding features of the medicament delivery device 500 as for the medicament delivery device 300.

In the medicament delivery device 500, the axially-extending component 319 is a first axially-extending component. A second axially-extending component 520 engages the first axially-extending component 319 when the cap 554 is attached to the medicament delivery device 500. This is in contrast to FIGS. 3A to 3C in which the proximally-extending portion of the cap engages the axially-extending component 319.

The medicament delivery device 500 has a cap 554 which is removably attached to the medicament delivery device, for example to the body 301. The cap 554 is configured to prevent movement of the actuation member 327 from the first position to the second position when the cap 554 is attached to the medicament delivery device 500.

The cap 554 has a proximally-extending portion 518 which is configured to prevent movement of the actuation member 327 from the first position to the second position.

The radially-extending surface 320 is configured to engage the first axially-extending component 319 for preventing distal movement of the axially-extending component 319.

The second axially-extending component 520 is axially movable relative to the body 301. The second axially-extending component 520 is configured to engage the first axially-extending component 319. The second axially-extending component 520 is configured to engage the first axially-extending component 319 on the opposite side of the first axially-extending component 319 to the radially-extending surface 320. If the first axially-extending component 319 has a protrusion 322 then the second axially-extending component 520 is configured to engage the first axially-extending component 319 on the opposite side to the protrusion 322.

The proximally-extending portion 518 is configured to engage the second axially-extending component 520 when the cap 554 is attached to the medicament delivery device 500.

The proximally-extending portion 518 has a protrusion 522 and the second axially-extending component 520 has a recess 521. The protrusion 522 is received in the recess 521 when the cap 554 is attached to the medicament delivery device and the cap 544 is in a first position as shown, for example in FIG. 7A. When the cap is in the first position then the actuation member 327 is prevented from moving distally from the first position to the second position.

The medicament delivery device 500 has a blocking surface 523 which is configured to engage the proximally-extending portion 518 on the opposite side of the proximally-extending portion 518 to the protrusion 522 when the cap 554 is attached to the medicament delivery device 500. The proximally-extending portion 518 is flexible. The blocking surface 523 engages the proximally-extending portion 518 to prevent the proximally-extending portion 518 from flexing. The blocking surface 523 is axially fixed in relation to the body 301. The blocking surface 523 is configured to maintain the protrusion 522 in the recess 521 when the cap 554 is attached to the medicament delivery device. The second axially-extending component 520 is axially movable with the proximally-extending portion 518 when the protrusion 522 is received in the recess 521. The second axially-extending component 520 is axially coupled to the proximally-extending portion 518.

The blocking surface 523 engages the proximally-extending portion 518 when the cap 554 moves moved a first distance in the distal direction from the first position to an intermediate position as shown, for example, in FIG. 7B.

When the cap 554 moves from the first position to the intermediate position then the second axially-extending component 520 moves distally with the proximally-extending portion 418. This is due to the protrusion 522 engaging the recess 521. The protrusion 522 is maintained in the recess 521 by the blocking surface 523.

The blocking surface 523 has an axial length which is limited such that the proximally-extending portion 518 is disengaged from the blocking surface 523 when the cap 554 moves distally from the intermediate position as shown, for example, in FIG. 7C.

The proximally-extending portion 518 is configured to flex when the cap 554 moves distally from the intermediate position as shown, for example, in FIG. 5C. The proximally-extending portion 518 is configured to flex when the protrusion 522 disengages the recess 521. The cap 554 can then be fully removed from the medicament delivery device 500 as shown, for example, in FIG. 7D. The protrusion 522 disengages the recess 521 when the cap moves distally from the intermediate position. The second axially-extending component 520 is no longer axially coupled to the proximally-extending portion 518 when the cap moves distally from the intermediate position.

The recess 521 and/or the protrusion 522 has a contoured surface for allowing the protrusion 522 to disengage the recess 521. The contoured surface may comprise a curved surface and/or a planar surface at an acute angle to the longitudinal axis of the medicament delivery device. In the example shown in FIGS. 7A to 7C, the protrusion 522 has the contoured surface 524.

When the protrusion 522 disengages the recess 521 then the second axially-extending component 520 no longer moves axially with the cap 554.

The cap 554 is configured to move the second axially-extending component 520 to disengage the first axially-extending component 519 as shown, for example, in FIG. 5C. The second axially-extending component 520 disengages the first axially-extending component 319 when the cap 554 moves distally from the intermediate position. When the second axially-extending component 520 is disengaged from the first axially-extending component 319 then the first axially-extending component 319 is permitted to move distally. The actuation member is therefore able to be moved from the first position to the second position for dispensing medicament from the device.

The first axially-extending component 319, the second axially-extending component 520 and the proximally-extending portion 318 can be seen to constitute a first set. In another embodiment, there may be multiple sets each having a first axially-extending component, a second axially-extending component and a proximally-extending portion as described and/or contemplated herein.

The medicament delivery device 500 has a dispensing mechanism 229 which is configured to dispense medicament from the needle 217. The actuation member 327 is configured to release the dispensing mechanism 229, for example by engaging the dispensing mechanism, when the actuation member 327 is in the second position.

As noted above, the dispensing mechanism 229 may have alternative or additional features to those described and/or contemplated in relation to FIGS. 2A to 2G. The dispensing mechanism may have features as described and/or contemplated herein, for example in relation to FIGS. 1A and 1B.

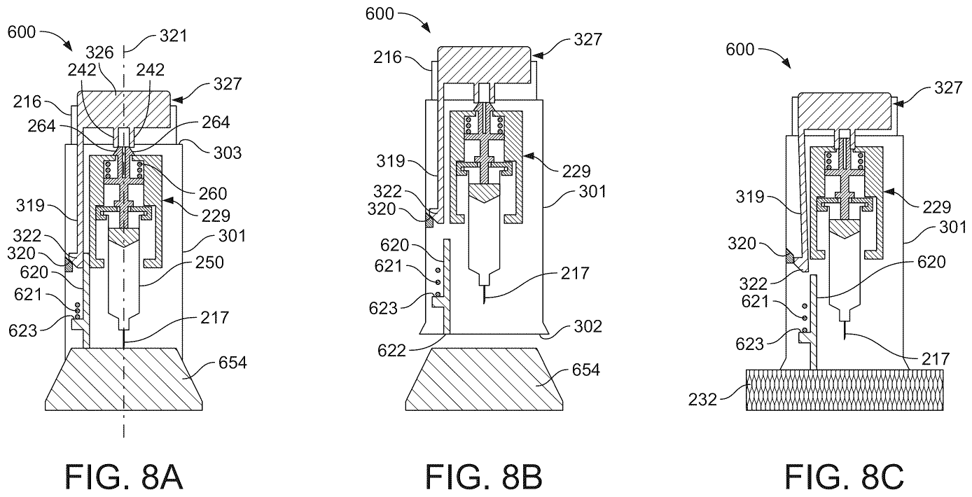

FIGS. 8A to 8C show a schematic view of parts of a medicament delivery device 600 in accordance with an embodiment of the disclosure. The medicament delivery device 600 has corresponding features as described and/or contemplated herein in relation to the medicament delivery device 300. The same reference numbers are used to annotate corresponding features of the medicament delivery device 600 as for the medicament delivery device 300.

In the medicament delivery device 600, the axially-extending component 319 is a first axially-extending component. A second axially-extending component 620 engages the first axially-extending component 319 when the cap 654 is attached to the medicament delivery device 600. This is in contrast to FIGS. 3A to 3C in which the proximally-extending portion of the cap engages the axially-extending component 319.

The cap 654 is removably attached to the medicament delivery device, for example to the body 301. The cap 654 is configured to prevent movement of the actuation member 327 from the first position to the second position when the cap 654 is attached to the medicament delivery device 600.

The cap 654 is configured to engage a distal-facing surface 622 of the second axially-extending component 620 to prevent the second axially-extending component 620 from moving distally when the cap 654 is attached to the medicament delivery device 600. The distal-facing surface 622 may be provided on a free end of the second axially-extending component or the distal-facing surface 622 may be proximally positioned from the free end of the second axially-extending component 620. The distal-facing surface 622 may be orthogonal to the longitudinal axis 321 of the medicament delivery device. In another embodiment the distal-facing surface 622 is at an acute angle to the longitudinal axis 321.

The medicament delivery device 600 has a bias 621 in the form of a spring which is configured to bias the second axially-extending component 620 in the distal direction. The bias 621 is configured to engage an engagement surface 623 on the second axially-extending component 620.

The radially-extending surface 320 is configured to engage the first axially-extending component 319 for preventing distal movement of the first axially-extending component 319 as described and/or contemplated herein. When the second axially-extending component 620 engages the first axially-extending component 319 then the first axially-extending component 319 is prevented from moving distally. The actuation member 327 is therefore prevented from being moved from the first position to the second position.

The second axially-extending component 620 is axially movable relative to the body 301. The second axially-extending component 620 is configured to engage the first axially-extending component 319. The second axially-extending component 620 is configured to engage the first axially-extending component 319 on the opposite side of the first axially-extending component 319 to the radially-extending surface 320. If the first axially-extending component 319 has a protrusion 322 then the second axially-extending component 620 is configured to engage the first axially-extending component 319 on the opposite side to the protrusion 322.

The cap 654 is configured to engage the second axially-extending component 620 when the cap 554 is attached to the medicament delivery device 600 as shown, for example, in FIG. 8A. The second axially-extending component 620 is prevented from moving distally when the cap 654 is attached to the medicament delivery device.

When the cap 654 is removed from the medicament delivery device then the second axially-extending component 620 moves distally under the force of the bias 621.

The second axially-extending component 620 disengages the first axially-extending component 319 when second axially-extending component moves distally as shown, for example, in FIG. 8B. When the second axially-extending component 620 is disengaged from the first axially-extending component 319 then the first axially-extending component 319 is permitted to move distally. The actuation member 327 is therefore able to be moved from the first position to the second position as shown, for example, in FIG. 8C.

The first axially-extending component 319 and the second axially-extending component 620 can be seen to constitute a first pair. In another embodiment, there may be multiple pairs each having a first axially-extending component and a second axially-extending component as described and/or contemplated herein.

The medicament delivery device 600 has a dispensing mechanism 229 which is configured to dispense medicament from the needle 217. The actuation member 327 is configured to release the dispensing mechanism 229, for example by engaging the dispensing mechanism, when the actuation member 327 is in the second position.

As noted above, the dispensing mechanism 229 may have alternative or additional features to those described and/or contemplated in relation to FIGS. 2A to 2G. The dispensing mechanism may have features as described and/or contemplated herein, for example in relation to FIGS. 1A and 1B.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases.

Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB 28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(w-carboxyheptadecanoyl)-des(B30) human insulin and B29-N-(w-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin. Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV). The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, small modular immunopharmaceuticals (SMIP), binding-domain immunoglobulin fusion proteins, camelized antibodies, and immunoglobulin single variable domains. Additional examples of antigen-binding antibody fragments are known in the art.

The term “immunoglobulin single variable domain” (ISV), interchangeably used with “single variable domain”, defines immunoglobulin molecules wherein the antigen binding site is present on, and formed by, a single immunoglobulin domain. As such, immunoglobulin single variable domains are capable of specifically binding to an epitope of the antigen without pairing with an additional immunoglobulin variable domain. The binding site of an immunoglobulin single variable domain is formed by a single heavy chain variable domain (VH domain or VHH domain) or a single light chain variable domain (VL domain). Hence, the antigen binding site of an immunoglobulin single variable domain is formed by no more than three CDRs.

An immunoglobulin single variable domain (ISV) can be a heavy chain ISV, such as a VH (derived from a conventional four-chain antibody), or VHH (derived from a heavy-chain antibody), including a camelized VH or humanized VHH. For example, the immunoglobulin single variable domain may be a (single) domain antibody, a “dAb” or dAb or a Nanobody® ISV (such as a VHH, including a humanized VHH or camelized VH) or a suitable fragment thereof. [Note: Nanobody® is a registered trademark of Ablynx N.V.]; other single variable domains, or any suitable fragment of any one thereof. “VHH domains”, also known as VHHs, VHH antibody fragments, and VHH antibodies, have originally been described as the antigen binding immunoglobulin variable domain of “heavy chain antibodies” (i.e., of “antibodies devoid of light chains”; Hamers-Casterman et al. 1993 (Nature 363:446-448). The term “VHH domain” has been chosen in order to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHH's, reference is made to the review article by Muyldermans 2001 (Reviews in Molecular Biotechnology 74:277-302).

For the term “dAb's” and “domain antibody”, reference is for example made to Ward et al. 1989 (Nature 341:544), to Holt et al. 2003 (Trends Biotechnol. 21:484); as well as to WO 2004/068820, WO 2006/030220, WO 2006/003388. It should also be noted that, although less preferred in the context of the present invention because they are not of mammalian origin, single variable domains can be derived from certain species of shark (for example, the so-called “IgNAR domains”, see for example WO 2005/18629).

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1: 2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container. As further described in ISO 11608-1: 2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user). As further described in ISO 11608-1: 2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1: 2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation). An example of a compound to be administered with the drug delivery device disclosed herein is a compound with the INN tirzepatide, as referenced in claim 1 of U.S. Pat. No. 9,474,780.

An example of a pharmaceutical composition to be administered with the drug delivery device disclosed herein is a pharmaceutical composition as referenced in U.S. Pat. No. 11,357,820.

An example of a pharmaceutical composition to be administered with the drug delivery device disclosed herein includes a 0.5 mL solution of 2.5 mg, 5 mg, 7.5 mg, 10 mg, 12.5 mg, or 15 mg of tirzepatide and the following excipients sodium chloride (4.1 mg), sodium phosphate dibasic heptahydrate (0.7 mg), and water for injection. Hydrochloric acid solution and/or sodium hydroxide solution may be added to adjust the pH.

An example starting dosage tirzepatide may be 2.5 mg injected subcutaneously once weekly. After four weeks, the tirzepatide dosage may be increased to 5 mg injected subcutaneously once weekly. The dosage may be further increased in 2.5 mg increments after at least four weeks on the current dose. In an example, the maximum dosage of tirzepatide may be 15 mg injected subcutaneously once weekly. If a dose is missed, patients may be instructed to administer tirzepatide as soon as possible within four days (96 hours) after the missed dose. If more than four days have passed, patients may skip the missed dose and administer the next dose on the regularly scheduled day. In each case, patients may then resume their regular once weekly dosing schedule. The day of weekly administration may be changed, if necessary. The time between two doses may be at least three days (72 hours).

Tirzepatide dosages may include 2.5 mg/ 0.5 mL, 5 mg/ 0.5 mL, 7.5 mg/ 0.5 mL, 10 mg/ 0.5 mL, 12.5 mg/ 0.5 mL, and 15 mg/ 0.5 mL. Tirzepatide may be stored in a refrigerator at 2° C. to 8° C. (36° F. to 46° F.). A single-dose pen or single-dose vial may be stored unrefrigerated at temperatures not to exceed 30° C. (86° F.) for up to 21 days. Tirzepatide may be stored in a carton.

### LIST OF FEATURES

10—Device
12—cap
13—needle sleeve
17—needle
20—distal region
21—proximal region
22—button
23—piston
200—medicament delivery device
201—body
202—distal end of the body
208—locking member
216—lock ring
217—needle
223—plunger
227—actuation member
228—button
229—dispensing mechanism
232—injection site
240—spring guide
242—protrusions
250—syringe
254—cap
258—stop
260—spring
262—spring
264—clip
265—proximal opening
266—needle shield
267—collar
268—collar
300—medicament delivery device
301—body
302—distal end of body
303—proximal end of body
318—proximally-extending portion
319—axially-extending component
319B—axially-extending component
320—radially-extending surface
321—longitudinal axis
322—protrusion
323—exterior side of axially-extending component
324—interior side of axially-extending component
325—free end of the axially-extending component
326—button
327—actuation member
328—proximal end of the medicament delivery device
354—cap
370—medicament delivery device
400—medicament delivery device
418—proximally-extending portion
419—axially-extending component
419B—axially-extending component
426—button
427—actuation member
428—distal-facing surface
429—free end
430—proximal-facing surface
431—free end
432—axially-extending arm
433—axially-extending arm
454—cap
470—medicament delivery device
500—medicament delivery device
301—body
302—distal end of body
303—proximal end of body
518—axially-extending portion
520—axially-extending component
521—recess
522—protrusion
523—blocking surface
545—cap
600—medicament delivery device
620—axially-extending component
621—bias
622—distal-facing surface
623—engagement surface
654—cap

1-20. (canceled)

21. A medicament delivery device for injecting medicament, wherein the medicament delivery device comprises:
a body having (i) a proximal end and a distal end defining a longitudinal axis, and (ii) a radially-extending surface oriented at an acute angle to the longitudinal axis of the body and directly contacted with and fixed to an inner surface of the body;
a needle;
an actuation member movable relative to the body from a first position to a second position for dispensing the medicament via the needle, wherein the actuation member comprises a first axially-extending component that is deflectable in a radially inward direction and configured to engage the radially-extending surface when the actuation member is in the first position, and wherein the radially-extending surface is fixed to the body when the actuation member moves relative to the body from the first position to the second position;
a cap removably attached to the medicament delivery device, and a second axially-extending component configured to engage the first axially-extending component on a side opposite to the radially-extending surface along a transverse axis perpendicular to the longitudinal axis when the cap is attached to the medicament delivery device, thereby preventing movement of the actuation member from the first position to the second position.

22. The medicament delivery device according to claim 21, wherein the cap comprises a proximally-extending portion configured to prevent movement of the actuation member from the first position to the second position.

23. The medicament delivery device according to claim 22, further comprising an axially-extending component configured to engage the proximally-extending portion of the cap to prevent movement of the actuation member from the first position to the second position.

24. The medicament delivery device according to claim 23, wherein the actuation member comprises the axially-extending component.

25. The medicament delivery device according to claim 23, wherein the axially-extending component and the actuation member are separate components.

26. The medicament delivery device according to claim 21, wherein the radially-extending surface is configured to engage the first axially-extending component for preventing distal movement of the first axially-extending component.

27. The medicament delivery device according to claim 26, wherein the cap comprises a proximally-extending portion which is configured to engage the axially-extending component on the opposite side of the axially-extending component to the radially-extending surface.

28. The medicament delivery device according to claim 21, wherein the second axially-extending component is movable relative to the body.

29. The medicament delivery device according to claim 28, wherein the cap comprises a proximally-extending portion configured to engage the second axially-extending component when the cap is attached to the medicament delivery device.

30. The medicament delivery device according to claim 29, wherein one of the proximally-extending portion and the second axially-extending component comprises a protrusion and the other of the proximally-extending portion and the second axially-extending component comprises a recess, and wherein the cap is configured such that the protrusion is received in the recess when the cap is attached to the medicament delivery device.

31. The medicament delivery device according to claim 21, wherein the cap is configured to engage a distal-facing surface of the second axially-extending component to prevent the second axially-extending component from moving distally when the cap is attached to the medicament delivery device.

32. The medicament delivery device according to claim 21, further comprising a bias disposed within the body and configured to bias the second axially-extending component in the distal direction.

33. The medicament delivery device according to claim 21, wherein the first axially-extending component comprises a protrusion which extends radially from the first axially-extending component, and wherein the radially-extending surface is configured to engage the protrusion.

34. The medicament delivery device according to claim 21, wherein the first axially-extending component is flexible, and wherein the first axially-extending component is configured such that when the actuation member moves from the first position to the second position, the first axially-extending component moves distally and is deflected radially by the engagement of the first axially-extending component and the radially-extending surface.

35. The medicament delivery device according to claim 23, wherein the proximally-extending portion is configured to abut a distal-facing surface of the axially-extending component to prevent the actuation member from moving from the first position to the second position.

36. The medicament delivery device according to claim 21, wherein the actuation member comprises a button configured to be pressed to move the actuation member from the first position to the second position.

37. The medicament delivery device according to claim 21, wherein the device comprises a dispensing mechanism configured to dispense the medicament via the needle, and wherein the actuation member is configured to release the dispensing mechanism when the actuation member is in the second position.

38. The medicament delivery device according to claim 21, further comprising a container containing the medicament.

39. A method of preparing a medicament delivery device for use prior to dispensing a medicament from the medicament delivery device, the method comprising:
removing a cap from the medicament delivery device, wherein the cap is configured to prevent movement of an actuation member of the medicament delivery device from a first position to a second position when the cap is attached to the medicament delivery device,
wherein the medicament delivery device comprises:
a body having (i) a proximal end and a distal end defining a longitudinal axis, and (ii) a radially-extending surface oriented at an acute angle to the longitudinal axis of the body and directly contacted with and fixed to an inner surface of the body;
a needle;
the actuation member movable relative to the body from the first position to the second position for dispensing the medicament via the needle, wherein the actuation member comprises a first axially-extending component that is deflectable in a radially inward direction and configured to engage the radially-extending surface when the actuation member is in the first position, and wherein the radially-extending surface is fixed to the body when the actuation member moves relative to the body from the first position to the second position;
the cap; and
a second axially-extending component configured to engage the first axially-extending component on a side opposite to the radially-extending surface along a transverse axis perpendicular to the longitudinal axis when the cap is attached to the medicament delivery device, thereby preventing movement of the actuation member from the first position to the second position.

40. A method of using a medicament delivery device, the method comprising:
removing a cap from the medicament delivery device, wherein the cap is configured to prevent movement of an actuation member of the medicament delivery device from a first position to a second position when the cap is attached to the medicament delivery device; and
moving the actuation member from the first position to the second position for dispensing the medicament from the medicament delivery device,
wherein the medicament delivery device comprises:
a body having (i) a proximal end and a distal end defining a longitudinal axis, and (ii) a radially-extending surface oriented at an acute angle to the longitudinal axis of the body and directly contacted with and fixed to an inner surface of the body;
a needle;
the actuation member comprising a first axially-extending component that is deflectable in a radially inward direction and configured to engage the radially-extending surface when the actuation member is in the first position, and wherein the radially-extending surface is fixed to the body when the actuation member moves relative to the body from the first position to the second position;
the cap; and
a second axially-extending component configured to engage the first axially-extending component on a side opposite to the radially-extending surface along a transverse axis perpendicular to the longitudinal axis when the cap is attached to the medicament delivery device, thereby preventing movement of the actuation member from the first position to the second position.

41. The medicament delivery device of claim 21, wherein the second axially-extending component is disposed within the body.

42. The medicament delivery device of claim 32, wherein the second axially-extending component comprises an engagement surface and the bias is configured to engage the engagement surface to bias the second axially-extending component in the distal direction.
